(19) United States
(12) Patent Application Publication
(10) Pub. No.: US 2026/0283732 A1
(43) Pub. Date: Sep. 24, 2026

(54) SUPPORTS FOR SURGICAL INSTRUMENTS

(71) Applicant: Sure Count Surgical, LLC, Los Angeles, CA (US)

(72) Inventor: Lawrence M. Richman, MD, Los Angeles, CA (US)

(21) Appl. No.: 19/689,855

(22) Filed: May 27, 2026

Related U.S. Application Data
(63) Continuation of application No. 29/971,035, filed on Oct. 31, 2024.
Application No. 29/971,035 is a continuation of application No. 18/739,014, filed on Jun. 10, 2024.
Application No. 18/739,014 is a continuation of application No. 18/140,505, filed on Apr. 27, 2023, now Pat. No. 12,036,050.

(51) Int. Cl. A61B 50/33; A61B 90/00

(52) U.S. Cl. CPC A61B 50/33; A61B 2090/0804; A61B 2090/0807

(57) ABSTRACT

The device mounts rear supports and forward supports aligned with rear instrument on the base of a tray. A forward support holds the forward end of the instruments. The forward support has a pivoting bar over the instruments. The bar is horizontal when the forward support is full, and the bar pivots to below horizontal when an instrument is removed. The bar's position tells the surgeon and staff whether the forward support is full. A stop on the bar can contact structure on the forward support to limit positions of the bar.

### BACKGROUND

### 1. Field

Device for organizing surgical instruments for operating rooms.

### 2. Related Patents and Applications

This application is a continuation of U.S. patent application Ser. No. 29/971,035, filed Oct. 31, 2024, titled “Instrument Support Design,” which is a continuation of U.S. patent application Ser. No. 18/739,014, filed Jun. 10, 2024, titled “Management Systems for Surgical Instruments and Items Used During Surgery,” which is a continuation of U.S. patent application Ser. No. 18/140,505, filed Apr. 27, 2023, now U.S. Pat. No. 12,036,050, granted Jul. 16, 2024 and entitled “Management Systems for Surgical Instruments and Items Used During Surgery,” the entire content of which are incorporated by reference in this application.

### 3. General Background and State of the Art

Surgeons and their surgical team need well-organized and readily accessible instruments and other items. Looking through a pile of assorted forceps and other instruments for a particular sized forceps adds times to surgeries. Any added time is undesirable for the patient because delays increase the length of the surgery and excessive time under anesthesia, and it increases the risk of post-operative infection.

Increasing the time for each surgery also affects surgeons, other doctors, and staff, but increased time also affects the hospital or surgical center adversely. Delays for each surgical team compound other teams'delay, which also can delay later procedures. This is analogous to an airplane unable to reach is assigned gate to unload passengers because an airplane at the gate is delayed leaving. Some hospitals have had to add operating rooms to accommodate the extra time spent in existing operating rooms. These add to hospital costs.

After each surgery, the team commonly gathers all surgical instruments taken into the operating room even if they are not used during surgery and mounts them through the instruments'finger openings on a stringer, two parallel, spaced stainless steel rods. The stringers hold the instruments together for easier transport, but the instruments can be disorganized during the gathering. Before the instruments are moved to the sterilization processing department, the team sprays the instruments and stringers with disinfectant that kills microbes that might otherwise float freely into the hospital corridors. The microbes also can become airborne within the sterilization department.

Loading the instruments on stringers, removing them for sterilization, and then organizing them for other surgeries is time-consuming and inefficient. These activities also increase the risk of glove punctures. Instruments also may be dropped to the floor, which can damage them or, at minimum, require repeating sterilization.

Increased costs and decreased efficiency are not the only concerns. Each team must account for all instruments when the procedure begins and ends. As an illustration, if the team starts with a ten of one instrument and uses all but three, it must account for the seven used and the three unused. Locating any missing one may be difficult and time consuming, which adds to the procedure's duration. Costs add up when accounting for multiple surgeries, each operating room, and each hospital. Some estimates assume a $40 per minute cost for each operating room delay.

The managed surgical instruments must be sterile during use. The surgical team must determine whether instruments touch non-sterile areas or objects in the operating room. Applicant's system helps sterilizing instruments, organizes them together, and reduces inadvertent contamination in the operating room. Doing so is more efficient than sterilizing the instruments separately and then loading the instruments for each upcoming surgery.

High-temperature steam and ethylene oxide (EO) gas used to sterilize instruments could damage system parts, so choosing parts that the heat and substances will not damage is important.

Different surgical procedures, e.g., cardiac versus orthopedic, typically require different instruments, assorted sizes of similar instruments, and different amounts of the same instruments. Applicant's system aids with organizing, accounting, and chain of custody for the surgical instruments. It also avoids puncturing gloves by sharp instruments. The system allows setting up containers for each type of surgery with the desired number of instruments and without unnecessary instruments. When the containers are complete, they can be moved to the scheduled operating room knowing that none are left out and no extras reach the operating room.

### SUMMARY

Applicant's design helps the hospital staff to see accurately and quickly based on the angle of a pivot bar if instrument supports have the proper number of instruments or if instruments were left behind during surgery.

The system for holding surgical instruments includes a tray having a floor. The tray may have sidewalls and a cover extending to the sidewalls over the tray's floor. The trays and instrument supports may be aluminum or a metal alloy because of their corrosion resistance so that heat from steam and chemicals like ethylene oxide (EO) do not affect the supports adversely during sterilization. Aluminum trays are also light weight.

The tray has openings to let the steam and EO gas circulate around the surgical instruments. The sidewalls may have similar openings.

A lid is attached to the top tray to protect the system during transport and sterilization. U.S. Pat. No. 12,036,050 discusses how a lid attaches to a tray. The lid may have an array of openings for steam and EO gas to reach the trays below, but the lid also may be solid. Solid lids may have printing and identification information. The system can include more than one tray above the first tray, and the trays can have structure for joining trays. The trays and lid may have connections to hold them together until they are separated at the operating room.

Depending on the type and number of instruments or other surgical devices for each tray, pairs of rear and forward platform are organized in positions on the tray. The platforms are designed to support the rear and forward parts of instruments.

The rear part has rear upright instrument supports attached to the tray's floor, and each rear upright instrument holds the handles or back ends of a stack of surgical instruments. The rear upright instrument supports may be like those described in U.S. Pat. No. 12,036,050.

A forward instrument supports is spaced in front of each rear part for supporting the forward section of instruments. Each forward part includes a forward upright instrument support, which also is secured to the tray's floor. For many instruments, the forward upright instrument support is near the instrument's hinge (pivot point), but it may be spaced from the hinge especially for longer forceps or other instruments.

The forward upright instrument support included a foot, which attaches to the tray's floor. The forward upright instrument support includes upright member that is generally perpendicular to the foot and floor. The lower part of upright member extends the full width of the upright member. Two spaced-apart side members are integral with the lower part of the upright member and extend upward from the upright member's lower part above a platform. The instruments rest on the platform. The vertical position of each platform may vary depending on the number and thickness of the instruments supported by the forward upright instrument support.

The proximal end of a bar mounts for pivoting on one of the spaced-apart side members. The distal end of the bar extends to the other side member. When the forward upright instrument support has a stack of a predetermined number of instruments on the platform, the top of the stack of instruments supports the bar in a horizontal orientation. That orientation signals to the surgical team that the forward upright instrument support is filled with the proper number of instruments and isn't overly filled, and no instrument is inadvertently left out.

The sterilization staff gets the same signals when loading instruments in the forward upright instrument supports. The bar pivots upward to allow loading an uppermost instrument to the instrument stack. Each time the top instrument is removed from the stack, the bar pivots below the horizontal starting position to rest on what becomes the top instrument. When another instrument is removed from the stack, the bar pivots farther.

Numbers may be printed on the forward upright instrument support near the distal end of the bar. The number “1” may be positioned where the end of the bar drops when one instrument is removed from the forward upright instrument support. “2” is positioned where the end of the bar is when the second instrument is removed, and so on. Or, for supports holding four instruments, the “4” could be positioned with a full forward upright instrument support, and the bar could be near “1” when the support holds one instrument. The numbers would alert the operative team what instruments are in use in real time, what instrument(s) may be missing within a body cavity, under a drape, on the floor, or elsewhere.

An edge member on the bar can contact a surface on the forward upright instrument support at a particular position to prevent the bar from pivoting farther down to the next lower instrument. The edge member also can prevent the bar from pivoting open too far to interfere with adjacent forward upright instrument supports.

### BRIEF DESCRIPTION OF THE DRAWINGS

FIG. 1 is a perspective view of one level of applicant's surgical instruments supports and their arrangements.

FIG. 2 is a plan view of an arrangement of instruments and support.

FIG. 2 is a perspective view of another level of applicant's surgical instruments supports and their arrangements.

FIG. 3 is a perspective view of applicant's forward upright instrument support.

FIG. 4 is another perspective view of applicant's forward upright instrument support looking from the opposite angle of FIG. 3.

FIG. 5 is a front sectional of applicant's forward upright instrument support through plane 5-5 of FIG. 3 and holding four instruments.

FIG. 6 is a front sectional view of applicant's forward upright instrument support holding two instruments.

FIG. 7 is a front sectional of applicant's forward upright instrument support with the bar in an open position.

FIG. 8 is a perspective view of sight stacks of instruments mounted on a tray of instruments.

FIG. 9 is a perspective view of another tier for mounting two surgical tools with space to mount other tools.

FIGS. 10, 11, and 12 are perspective views, each holding a different number of surgical instruments. FIG. 10 holds 2, FIG. 11 holds three, and FIG. 12 holds six.

### DETAILED DESCRIPTION

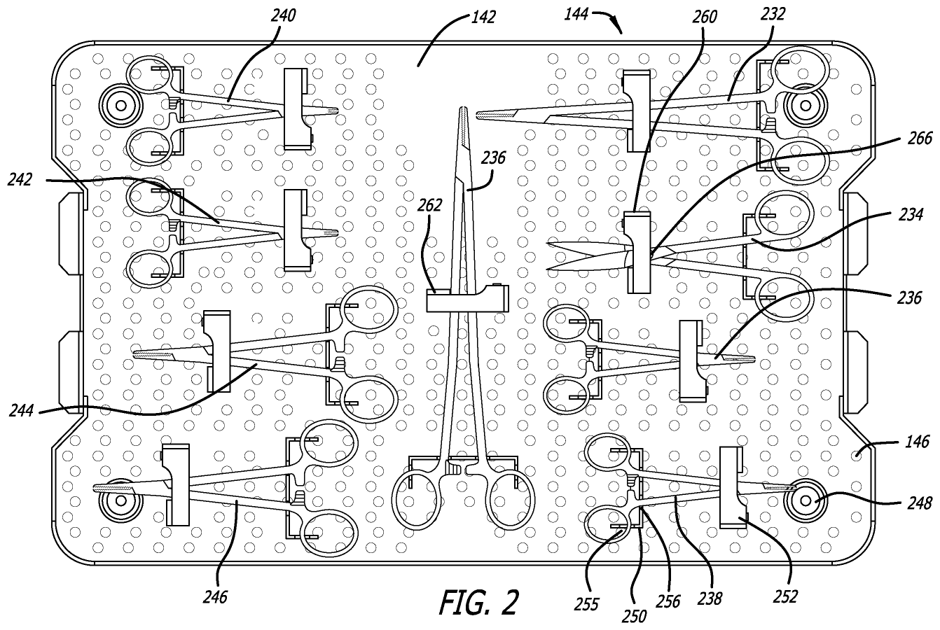

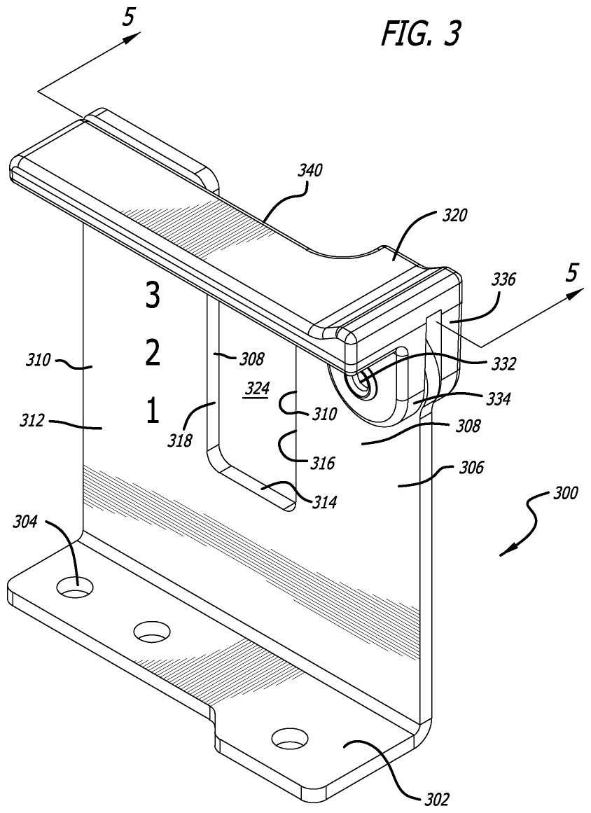

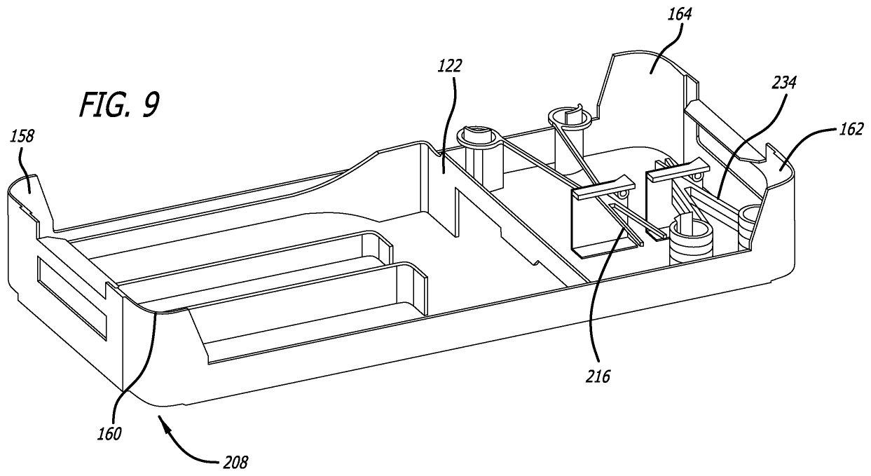

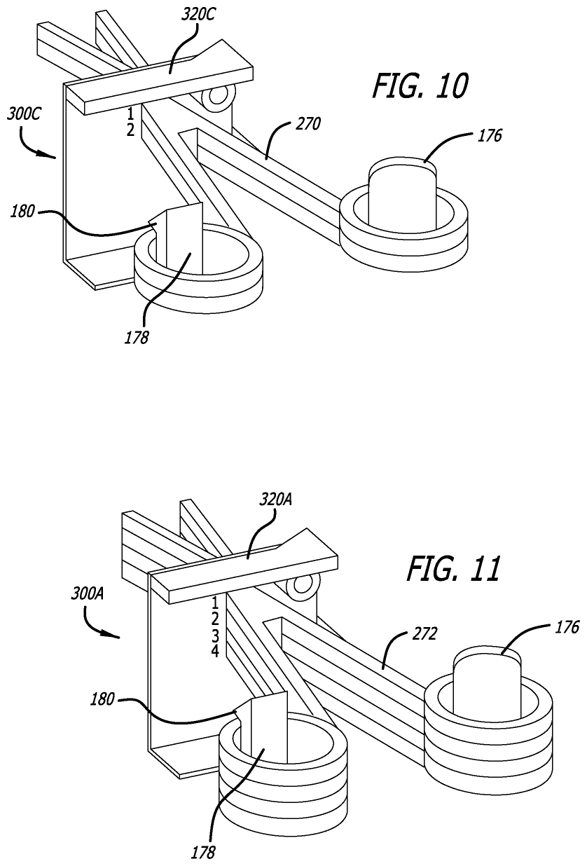

The elements of applicant's system mount on respective floors 102 and 202 of upper tray 100 (top tier) and bottom tray 200 (bottom tier) (FIGS. 8 and 9). See also floor 142 of tray 144 in FIG. 2, which has an array of openings 146. Upper tray 100 and bottom tray 200 also have arrays of openings but FIGS. 8 and 9 do not who the openings. The bottom tray's dividing wall 222 also has openings, FIG. 9 does not show. The openings may be circular or other shapes, and they may be arranged differently. The openings allow steam and EO to flow throughout the trays to the instruments and to the instrument supports.

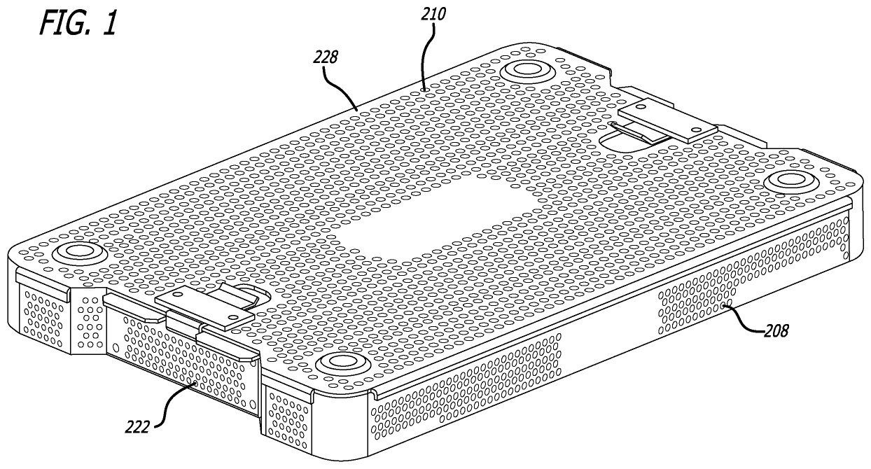

Lid 228 may attach to tray 100 (FIG. 1). The lid in FIG. 1 is perforated with openings to allow steam and EO to enter the trays, but the lid may be solid with printed instructions or identification. U.S. Pat. No. 12,036,050 discusses an attachment of the lid to the other trays.

Tray dimensions vary. At 51.5 mm long and 32.5 mm wide, tray 100 fits within a wide-body Aesculap® SterilContainer (see URL, accessed Mar. 30, 2026). Sidewall 20 measures 4.8 mm high. In FIG. 1, steam and ethylene oxide (EO) pass through openings 208, 210, and 222 to sterilize the instruments. Lid 228 features the most openings due to its large surface area, though floor 142 of tray 144 (FIG. 1) also has spaced openings 146. Designers can vary the size and number of these openings to optimize steam and EO flow.

The floor, sidewall and lid may be aluminum, but they may be aluminum, other metal alloys, or non-corrosive materials that heat and chemicals do not affect adversely during sterilization. Depending on the tray's height including its sidewall, mounting two trays together, one above another, may fit in Aesculap containers. Other manufacturers may make wider sterilization containers to accommodate the present system. Depending on the instruments loaded on the tray, a full tray weighs about 2 ¾ kg (≈6 lbs.).

FIGS. 8 and 9 show an arrangement for instruments that a surgeon may use for one devised surgical procedure. FIG. 8 shows eight instrument support combinations for holding instruments 110, 112, 114, 116, 118, 120, 122, and 124. The instruments mount above floor 102 of tray 100. FIG. 9 shows two instruments 214 and 216 mounted on tray 200, and the figure also has structure for other devices that could be used during surgery. Examples include electrocautery devices, bone saws, and drills. Different surgical procedures may use different numbers of surgical instruments, and the instruments may mount in different forward and rear supports and in different position to accommodate the chosen instruments.

Tray 102 has curved corners 150, 152 154 and 156 (FIG. 8). The corners may be spring loaded toward the outside of the tray. Tray 200 (FIG. 9) has four corners 158, 160, 162, and 164. Those corners can receive corners 150, 152 154 and 156 (FIG. 8). Spring loading of the corners for tray 100 against the corners of tray 200 help secure tray 100 above tray 200. One or both trays may have structure such as upward facing or downward facing feet for maintaining the position of tray 100 above tray 200.

The tray's floor may have printed identifications 128 (FIG. 8). The letters in FIG. 8 stand for instruments'names, sizes, quantities, and other information about a nearby stack of instruments should mount on the supports.

FIG. 2 shows an arrangement for instruments on tray 142. Instruments 230, 232, 234, 236. 238, 240. 242, 244, and 248 mount on rear support 250 and forward support 252. Tray 144 may have feet 240 to stabilize the tray on the surfaces on which the tray rests.

The rear supports 250 may be like those in U.S. Pat. No. 12,036,050. The width of each rear support may differ to accommodate different size instruments. Compare the rear support for the large instrument 230 with the narrower rear support for smaller instrument 238.

The rear supports are designed to make removing the instruments'handle ends during surgery, and loading sterile instrument in the proper rear support. The rear supports 170 in FIG. 8 have spaced-apart curved fingers 172 and 174 that conform to the finger rings of forceps 124. Having the fingers'curvature have the same curvature as the finger rings may be unnecessary, however.

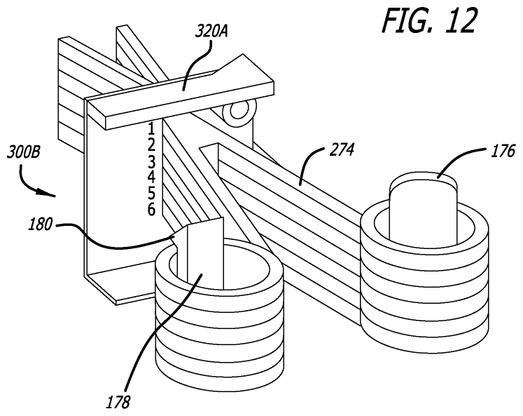

FIGS. 10, 11, and 12 show one curved finger 176 for one finger ring. Instead of a second curved finger, the other finger ring is held by an arm 178 with a hook 180. The arm and hook urge the rear ends of the instrument into a tight stack. The arm can be pivoted away from the finger rig's surface when removing an instrument from its forward support 300A, 300B, or 300C.

Rear support 250 in FIG. 2 differs from those in FIG. 8. It has two slots 256 and 258 on each side of the rear support. One finger ring of forceps 246 seats in slot 256, and the forceps'shank seats in slot 258. The rear support in the trays of FIGS. 8 and 9, can be used in the FIG. 2 tray, and the rear support in FIG. 2 can be used in the trays of FIGS. 8 and 9.

Forward supports 140 (FIG. 8) or 260 (FIG. 2) hold the forward part of instruments. The forward support for smaller instruments may be near the instrument's pivot point. See pivot point 266 at forward support 260 (FIG. 2). For longer instruments like forceps 238, the instrument's pivot point can be spaced farther forward of forward support 262 (FIG. 2).

Surgeons may require one or more trays like tray 100 (FIG. 8) or 144 (FIG. 2) depending on the planned surgical procedure. And the number of instruments for each rear/forward support combination can vary too. Different supports in FIGS. 10, 11, and 12 hold two instruments 270, four instruments 272, or six instruments 274.

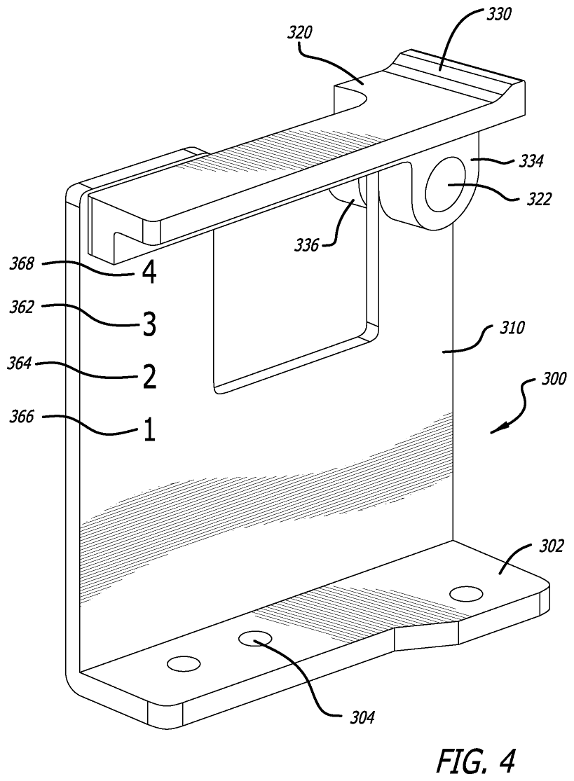

FIGS. 3 through 7 and 10 through 12 show details of the forward upright instrument support 300. Support 300A, 300B, and 300C (FIGS. 10-12) show variations. Referring to FIGS. 3-7, the forward upright instrument support has a base 302 that attaches to the tray's floor 102. The forward upright instrument support may be aluminum, an alloy of aluminum or other metal, or other non-corrosive materials that the heat and chemicals do not affect too adversely during sterilization. Base 302 may be welded to floor 102, chemically attached to the floor, or attached with fasteners like rivets or bolts (not shown) through openings 304 through the tray's floor.

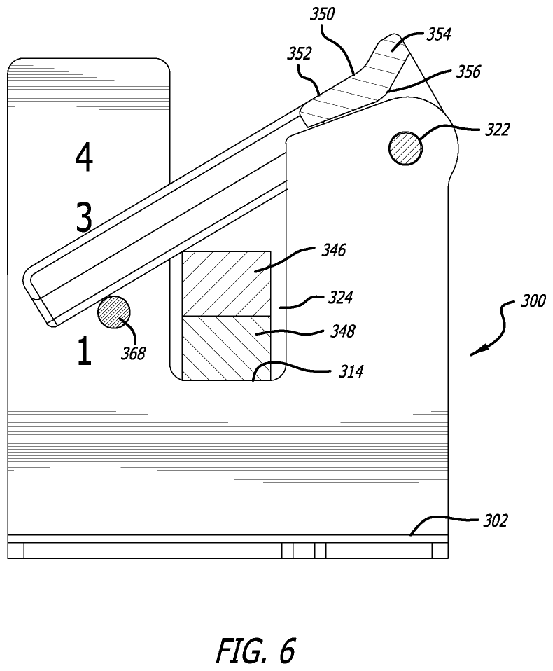

Forward upright instrument support also includes upright member 306 extending up from base 302. The upright member's lower portion 312 can extend the full width of the upright member (FIG. 5). Two spaced-apart side members 308 and 310 extend upward from the lower portion above platform 314. The spacing between the side members is wide enough to accommodate the part of the instruments that fits between the side members. Inside edges 316 and 318 of side walls 308 and 310 form the sides of cavity 324, the bottom of which is closed by platform 314. The platform is the surface on which the bottom surgical instrument rests when the forward upright instrument support holds at least one instrument. The distance of the platform above lower portion 312 may vary to accommodate stacks of different numbers and thicknesses of instruments. Compare FIGS. 10, 11, and 12. Though the platform in each figure is not visible, the figures show how the different platform heights affect the position of the bottom instrument.

The side wall edges 316 and 318 are vertical in the drawings, but they could taper inward from top to bottom if it makes loading instrument in cavity 324 easier. Platform 314 is planar in the drawings, but adding a small curvature with a lower center may be useful for supporting instruments especially if cavity 324 is narrow.

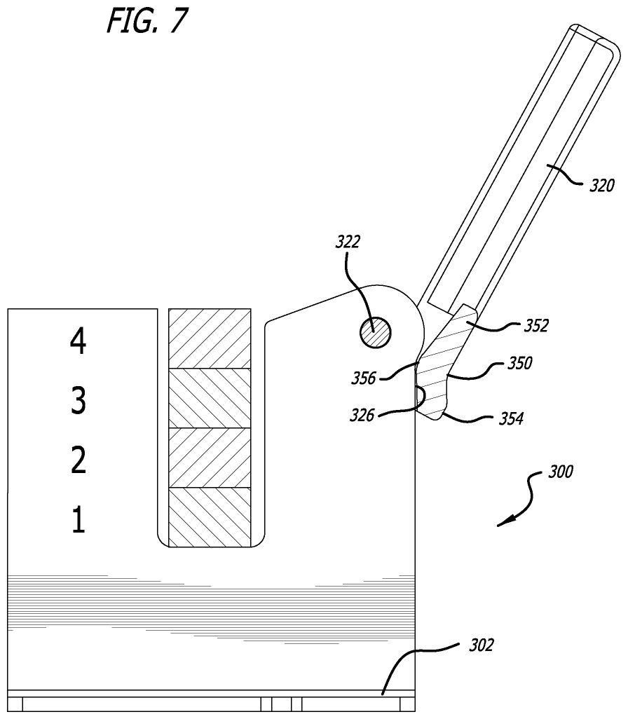

Bar 320 mounts for pivoting about an opening 322 through one side member 310 near the member's top. See FIGS. 3-7. The bar's proximal end 330 has two depending tabs 334 and 336 (FIGS. 5 and 6), which have openings aligned with the side member's opening. A fastener, which allows pivoting of the tabs, attaches on the outside of the tabs and extends through opening 322 in tab 324 (FIG. 5). Tab 336 also has an opening like opening 322 (not shown) for the other end of the fastener. The opening through tab 336 is not visible in the drawings.

Cutout region 340 of bar 320 allows the bar to pivot clockwise (FIG. 3) without side member 310 interfering.

Four instruments 342, 344, 346, and 348 (cross-section in FIG. 5) are stacked on platform 314 in cavity 324 of forward upright instrument support 300. If a cavity 324 must hold more or fewer instruments or instruments with different heights, the cavities'depths can change by choosing a different forward upright instrument support 300 with a higher or lower vertical position of the platform. See FIGS. 10, 11, and 12.

When the top two instruments are removed from the forward upright instrument support in FIG. 8, the corner of instrument 344 contacts bar 320 and prevents further bar counterclockwise movement. After removing third instrument 344 from cavity 324, which leave a single instrument 348 remaining in the cavity, the bar would continue counterclockwise pivoting, which may be undesirable. Edge member 350 mounted on bar 320 (FIG. 6) can stop further pivoting. The edge member has a forward end 352 and rear end 354 on the sides of rounded bottom 356. The edge member's curved bottom (FIG. 5) is out of contact with top 326 of upright side member 310. When bar 320 pivots counterclockwise (FIG. 6), the edge member's forward end 352 contacts the upright side member's top. That acts as a stop to block the bar's further counterclockwise pivoting.

As an alternative to edge member 350 acting as a stop for further counterclockwise rotation of bar 320, protrusion 358 may extend outward from side member 308 (FIG. 8) into the path of bar 320.

Edge member 350 is designed to position bar 320 at a desired orientation. Whether the bar is not horizontal because of the location of the topmost instrument 344 or because intersection of top surface 326 of side member 310 with edge member 350, a person sees that forward upright instrument support 300 is missing at least one instrument.

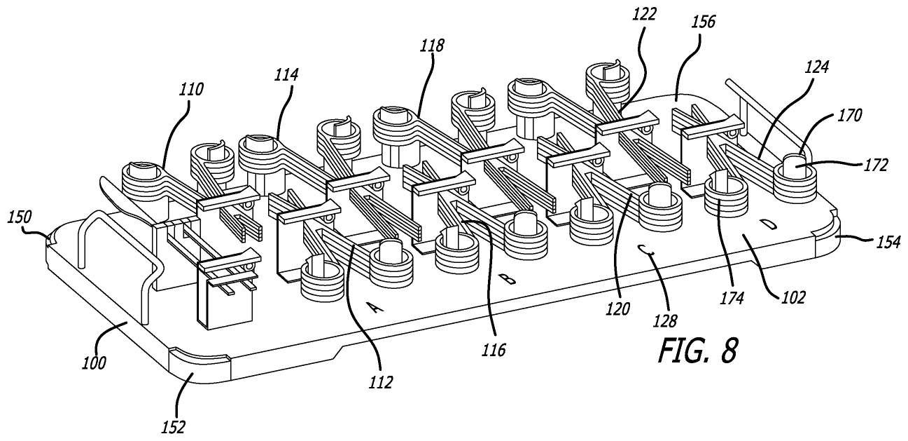

The height of platform 314 of forward upright instrument support 300 positions the top of topmost instrument 348. That position depends on the thickness of the instruments supported on the forward upright instrument support. The position of the top of topmost instrument causes bar 320 to be horizontal when the cavity has four instruments with dimensions like those in FIG. 7. Bars 320A, 320B, and 320C (FIGS. 10-12) are horizontal because each forward upright instrument support in each figure has the maximum number of instrument and is full. A horizontal bar signals to the surgical team and the hospital sterilizing staff that the forward upright instrument support is full, neither overfilled nor missing instruments.

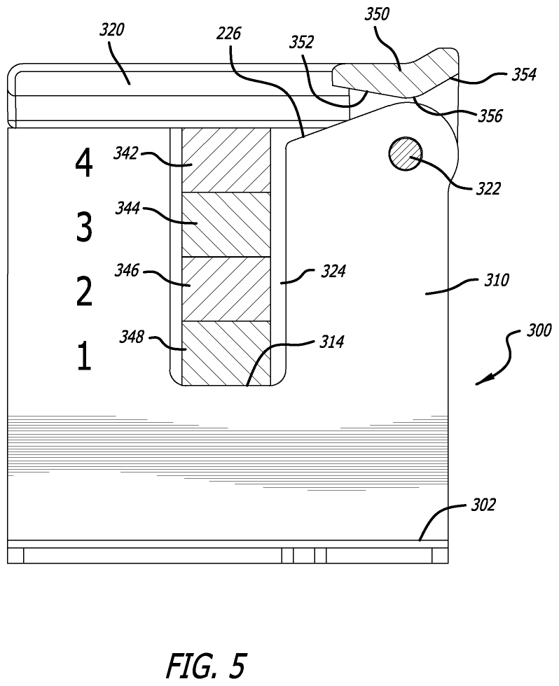

When instruments are removed from forward upright instrument support 300, they also are lifted off the corresponding rear instrument support like support 150 (FIG. 1) or 250 (FIG. 4).

Indicia 360, 362, 364, and 366 may be engraved on side member 308. The indicia correspond to positions of the bar's distal end to show how many instruments remain in forward upright instrument support 300. Rather than having “4” as the top number, “1” could be at the top, no number could be where “4” is, or “F” (for “full”) in the drawings. But the number of instruments remaining may be less important to the surgical team or the sterilizing department than the fact that at least one instrument has been removed during surgery or that the sterilizing department must add at least one instrument to refill the forward upright instrument support.

When refilling the front and rear instrument supports, bar 320 is pivoted clockwise to expose cavity 324 (FIG. 7). Rear end 354 of edge member 350 engages surface 326 of upright member 310 to function as a stop to limit the position of the bar. If the bar lacked the stop and was vertical or near vertical during refilling instead of its orientation is FIG. 9, the bar could fall counterclockwise more easily over cavity 324 to interfere with reloading. If the bar could continue pivoting clockwise, it might interfere with refilling other forward upright instrument supports.

As an alternative to having edge member 350 engaging surface 326 of upright member 310 to function as a stop, protrusion 368 mounts on side member 310 in the path of tab 334 and limits clockwise rotation of the bar. (FIG. 6). The protrusion also could mount of the other side of side member 310 to engage tab 336.

The description is illustrative, not limiting, and is for example only. Although this application shows and describes examples, those having ordinary skill in the art will find it apparent they can make changes. Many examples involve specific combinations of method acts or system elements, but those acts and those elements may be combined in other ways to meet the same goals. Acts, elements, and features discussed only with one embodiment are not intended to be excluded from a similar role in other embodiments.

“Plurality” means two or more. A “set” of items may include one or more of such items. The terms “comprising,” “including,” “carrying,” “having,” “containing,” “involving,” and similar words in the written description or the claims are open-ended—each means, “including but not limited to.” Only the transitional phrases “consisting of” and “consisting essentially of” are closed or semi-closed transitional phrases regarding claims. The ordinal terms like “first,” “second,” “third,” etc., in the claims don't by themselves connote any priority, precedence, or order of one claim element over another or the temporal order in which acts of a method are performed. Instead, they merely are labels to distinguish one claim element having a certain name from another element having a same name (but for the ordinal term's use). Alternatives like “or” include one or any combination of the listed items.

I claim:

1. A device for supporting and allowing removal and addition of instruments to a tray of which the instruments include a handle part and a part forward of the handle part, the device comprising:
a forward, upright instrument support configured to attach to the tray and positioned to receive the part of the instrument part forward of the handle, the forward, upright instrument support part having a top and a bottom,
the forward, upright instrument support comprises a platform, first and second side members spaced apart from the platform and extending above the platform, the platform being configured to support a part of at least one instrument forward of the handle part;
and a hinge mounted on the first side member of the forward, upright instrument support, a bar having a proximal end mounted for pivoting on the hinge, the distal end of the bar pivoting adjacent to the second side member of the forward, upright instrument support.

2. The device of claim 1 wherein the first and second side members form a cavity above the platform, the cavity being sized to receive a part of at least two instruments, and wherein the bar being positioned to contact the topmost instrument in the cavity.

3. The device of claim 1 further comprising indicia adjacent to the second side member of the forward, upright instrument support near the distal end of the bar.

4. The device of claim 1, wherein the first side member has a top, the device further comprising a projection mounted on the bar and having a first position out of contact with the first side member, and wherein the bar is adapted to pivot in a path between a first position parallel to the platform, and a second position along the cavity.

5. The device of claim 4 where the projection is out of contact with the top of first side member when the bar is in its first position, and wherein the projection contacts the top of the first side member to limit movement of the bar in the second position of the bar.

6. The device of claim 4 where the bar is adapted to pivot in a path along the cavity in which the projection contacts the first side member to limit further pivoting of the bar.

7. The device of claim 1, wherein the first side member has a wall facing the cavity, and the second side member has a wall facing the cavity, the wall on the first side member and the wall on the second side member extending from the platform.

8. The device of claim 1 further comprising a rear upright instrument support configured to attach to the tray spaced from the forward, upright instrument support and configured to receive the handle part.

9. The device of claim 1, further comprising a second side member of the at least one first upright member, the upright member of the at least one first upright member being positioned at a first end of the platform of the at least one first upright member and the second side member of the first platform being positioned at a second end of the first platform, and wherein the first and second side members of the at least one first upright member extending above the first platform.

10. A device for holding instruments comprising:
a forward, upright instrument support comprising an upright member, the upright member having receiving means configured to receiving a stack of instruments from a topmost instrument to a bottommost instrument,
bar means having a proximal end pivotally mounted on an upright member, the bar means having a distal end spaced from the first end; the bar means pivoting toward the instruments when a topmost instrument is removed from the receiving means; and
projection means mounted on the bar means and being positioned to contact the upright member when a predetermined number of instruments are removed from the receiving means to prevent further pivoting of the bar means.
